(19) United States
(12) Patent Application Publication
(10) Pub. No.: US 2026/0335255 A1
(43) Pub. Date: Oct. 29, 2026

(54) PHARMACEUTICAL COMPOSITIONS

(71) Applicant: Reena PATEL, Mumbai, Maharashtra (IN)

(72) Inventors: Reena PATEL, Mumbai, Maharashtra (IN); Dinesh Shantilal PATEL, Mumbai, Maharashtra (IN); Sachin Dinesh PATEL, Mumbai, Maharashtra (IN); Shashikant Prabhudas KURANI; Milind Vinayak SATHE, Mumbai, Maharashtra (IN); Surjyanarayan MANDAL

(21) Appl. No.: 19/112,422

(22) PCT Filed: Sep. 22, 2023
(86) PCT No.: PCT/IB2023/059391

§ 371 Date: Mar. 17, 2025

(30) Foreign Application Priority Data
Sep. 22, 2022 (IN) 202221054235

(51) Int. Cl. A61K 31/135; A61K 9/00; A61K 9/08; A61K 47/02; A61K 47/10; A61K 47/18; A61K 47/32

(52) U.S. Cl. CPC A61K 31/135; A61K 9/0019; A61K 9/08; A61K 47/02; A61K 47/10; A61K 47/183; A61K 47/186; A61K 47/32

(57) ABSTRACT

The present invention provides subcutaneous pharmaceutical injectable compositions comprising ketamine or its salt such as Ketamine Hydrochloride equivalent to Ketamine base 50 mg/ml and stabilizer. The invention further provides a method for preparing the said compositions. Compositions of the present invention depict a typical pharmacokinetic profile. Compositions of the present invention are bioequivalent to currently marketed injectable compositions but have lower Cmax, similar Tmax and similar AUC. Compositions of the present invention are useful in treatment of treatment resistant depression and in management of pain.

### FIELD OF INVENTION

Present invention relates to subcutaneous injectable compositions comprising Ketamine equivalent to Ketamine base 50 mg/ml and a stabilizer. The compositions exhibits altered Cmax but similar Tmax and similar Area under curve (AUC). Invention also relates to the process to prepare subcutaneous injectable compositions comprising Ketamine and a stabilizer.

### BACKGROUND OF INVENTION

Ketamine Hydrochloride in the form of injection is used as a general anesthetic to prevent pain and discomfort during medical tests or procedures, or minor surgery. Several applications of Ketamine and its salts are being tried in several clinical trials. It is of great relief to patients suffering from Complex regional pain syndrome i.e. a poorly understood condition where a person experiences persistent severe and debilitating pain and in management of pain. Pain Management Specialists rely on Ketamine for Palliative care, to treat Oncology Chronic pain and such other pains. It is useful to treat treatment resistant depression. This wonder drug has several other applications. But it is also associated with some side effects. Common known side effects of ketamine hydrochloride include blurred vision, double vision, dream-like feeling, dizziness, drowsiness, jerky muscle movements, loss of appetite, nausea, vomiting, sleep problems such as insomnia. Other side effects include hallucinations, severe confusion and extreme fear unusual thoughts and alike.

Hospitalized patients or other patients are often prescribed some other medicines as well. These other medicines when consumed by patients also impact on effects produced by Ketamine or its salt. Ketamine may interact with barbiturates and other narcotics if administered to the patient. The presence of Ketamine or its quantity in blood after it is injected is somewhat unpredictable.

Ketamine or Ketamine Hydrochloride is a potent molecule and is administered under a physician's supervision. The initial dose of ketamine administered intravenously varies from 1 mg/kg to 4.5 mg/kg. The average amount of 2 mg/kg produces surgical anesthesia of about five to ten minutes. The initial dose of ketamine administered intramuscularly (IM) varies from 6.5 to 13 mg/kg. A dose of 10 mg/kg normally produces surgical anesthesia of about 12 to 25 minutes. Ketamine injection is presently available in strengths of 10 MG BASE/ML 50 MG BASE/ML 100 MG BASE/ML. Its solubility in water is 200 mg/ml.

Ketamine is racemic mixture of 2 isomers and S(+) ketamine has been shown to be significantly shorter acting than racemic ketamine, which allows a faster wake-up after use as an anesthetic and easier titration when used for sedation and/or analgesia. It is also known that single-shot systemic administration has a short-term effect only. [Pediatric Anesthesia 2005 15: 91-97, CHARLES LIN MD AND MARCEL E. DURIEUX MD PhD, Department of Anesthesiology, University of Virginia Health System Charlottesville, Charlottesville, VA, USA]

A single subanesthetic dose of ketamine reduces depressive symptoms. Patients experience transient dissociation and psychotomimetic side effects and hemodynamic changes that limit its clinical use.

Ketamine is known to have direct effects on free recall and recognition memory. Ketamine is known to produce significant levels of thought disorder and withdrawal-retardation in healthy volunteers. Ketamine is known to cause Changes in behavior and perception, acute perceptual and cognitive disturbances.

Label of Ketalar injection states that it is formulated as a slightly acid (pH 3.5-5.5) sterile solution for intravenous or intramuscular injection in concentrations containing the equivalent of either 10, 50 or 100 mg ketamine base/milliliter and contains not more than 0.1 mg/mL Phemerol® (benzethonium chloride) added as a preservative. 10 mg/mL solution has been made isotonic with sodium chloride.

There is a special note that states “SPECIAL NOTE EMERGENCE REACTIONS HAVE OCCURRED IN APPROXIMATELY 12 PERCENT OF PATIENTS. THE PSYCHOLOGICAL MANIFESTATIONS VARY IN SEVERITY BETWEEN PLEASANT DREAM-LIKE STATES, VIVID IMAGERY, HALLUCINATIONS, AND EMERGENCE DELIRIUM. IN SOME CASES THESE STATES HAVE BEEN ACCOMPANIED BY CONFUSION, EXCITEMENT, AND IRRATIONAL BEHAVIOR WHICH A FEW PATIENTS RECALL AS AN UNPLEASANT EXPERIENCE. THE DURATION ORDINARILY IS NO MORE THANA FEW HOURS; IN A FEW CASES, HOWEVER, RECURRENCES HAVE TAKEN PLACE UP TO 24 HOURS POSTOPERATIVELY. NO RESIDUAL PSYCHOLOGICAL EFFECTS ARE KNOWN TO HAVE RESULTED FROM USE OF KETALAR. THE INCIDENCE OF THESE EMERGENCE PHENOMENA IS LEAST IN THE ELDERLY (OVER 65 YEARS OF AGE) PATIENT. ALSO, THEY ARE LESS FREQUENT WHEN THE DRUG IS GIVEN INTRAMUSCULARLY AND THE INCIDENCE IS REDUCED AS EXPERIENCE WITH THE DRUG IS GAINED. THE INCIDENCE OF PSYCHOLOGICAL MANIFESTATIONS DURING EMERGENCE, PARTICULARLY DREAM-LIKE OBSERVATIONS AND EMERGENCE DELIRIUM, MAY BE REDUCED BY USING LOWER RECOMMENDED DOSAGES OF KETALAR IN CONJUNCTION WITH INTRAVENOUS DIAZEPAM DURING INDUCTION AND MAINTENANCE OF ANESTHESIA. (See DOSAGE AND ADMINISTRATION Section.) ALSO, THESE REACTIONS MAYBE REDUCED IF VERBAL, TACTILE, AND VISUAL STIMULATION OF THE PATIENT IS MINIMIZED DURING THE RECOVERY PERIOD. THIS DOES NOT PRECLUDE THE MONITORING OF VITAL SIGNS. IN ORDER TO TERMINATE A SEVERE EMERGENCE REACTION, THE USE OF A SMALL HYPNOTIC DOSE OF A SHORT-ACTING OR ULTRA SHORT-ACTING BARBITURATE MAYBE REQUIRED. WHEN KETALAR IS USED ON AN OUTPATIENT BASIS, THE PATIENT SHOULD NOT BE RELEASED UNTIL RECOVERY FROM ANESTHESIA IS COMPLETE AND THEN SHOULD BE ACCOMPANIED BY A RESPONSIBLE ADULT.”

WO2020232274 which discloses ketamine formulation for subcutaneous injection describes complexing with betacyclodextrins. WO/2022/109050 also describes complexing of ketamine with cyclodextrins.

EP3932393 claims a liquid pharmaceutical formulation comprising i). an active ingredient selected from ketamine and esketamine or a pharmaceutically acceptable salt thereof; ii). a cellulose ether; iii). glycerol; and iv) water.

Conventionally and currently available injections often exhibit one or more of side effects such as blurred vision, double vision, dream-like feeling, dizziness, drowsiness, jerky muscle movements, loss of appetite, nausea, vomiting, sleep problems such as insomnia and hence fullest medicinal potential of Ketamine or its salt such as hydrochloride salt is not fully realized. These side effects are due to sudden rise in Ketamine levels in the blood after administering injectable composition. An injection of ketamine or its salt such as hydrochloride salt that is free from one or more side effects or undesired effects is not available. Thus, there is urgent need to provide an injectable composition of Ketamine or its salt such as hydrochloride salt that is safe and effective and that shall have some predictable blood levels. Mere reduction of quantity of Ketamine in injectable composition would result into sub-therapeutic concentrations and therefore will not elicit desired medicinal effect while trying to get rid of undesirable effects. Thus there is a void of subcutaneous injectable compositions that do not cause undesirable sudden rise or spike of Ketamine after administering injectable compositions but produce Ketamine concentration to ensure therapeutic efficacy in patients with depression which exhibit Cmax above concentration needed for therapeutic efficacy.

There is a need to provide suitable form of injection such as subcutaneous injection for treatment of treatment resistant depression and for the treatment or management of chronic pains.

### OBJECTS OF THE INVENTION

In view of the foregoing, it is an object of the invention to provide an injectable composition comprising Ketamine equivalent to Ketamine base 50 mg/ml which is safer and free from the side effects resulting due to sudden rise in Ketamine concentration in blood after administration of conventional injectable dosage form.

It is another object of the invention to provide subcutaneous injectable compositions comprising Ketamine equivalent to Ketamine base 50 mg/ml that shall exhibit AUC similar to the currently marketed injectable compositions although it may have different Cmax, preferably lower Cmax and similar Tmax.

It is yet another object of the invention to provide a composition of Ketamine for treatment of treatment resistant depression and/or pain management.

It is still another object of the invention to provide a process for preparation of composition comprising Ketamine equivalent to Ketamine base 50 mg/ml which is safer and free from the side effects resulting due to sudden rise in Ketamine concentration in blood after administration of conventional injectable dosage form.

Yet another object of the invention is to provide an injectable composition comprising Ketamine and a stabilizer that exhibit some predictable blood levels.

Still another object of the invention is to provide subcutaneous injectable compositions comprising Ketamine equivalent to Ketamine base 50 mg/ml which exhibit Mean Cmax which is well above the concentration needed for therapeutic efficacy in patients with depression (0.5 mg/kg; 40 min infusion; 185 ng/ml), though it exhibits Cmax lower than currently marketed compositions.

### BRIEF DESCRIPTION OF FIGURE

FIG. 1 provides a graph of blood concentrations of Ketamine in nanograms (ng)/ml on Y axis from Test and Reference compositions against time in hours on X axis.

### SUMMARY OF THE INVENTION

The present invention provides an injectable composition comprising Ketamine equivalent to Ketamine base present as 50 mg/ml hat exhibits AUC similar to that is exhibited by presently marketed injectable compositions although it may have different Cmax, preferably lower Cmax and similar Tmax. The composition unexpectedly controls sudden rise of Ketamine in blood after being administered. The composition is an aqueous composition.

The composition of present invention is free from the side effects resulting due to sudden rise in Ketamine concentration in blood after administration of conventional injectable dosage form. The composition is a subcutaneous injectable composition.

The composition has a Cmax which is 80-100% of Cmax of marketed injectable compositions for subcutaneous use and AUC₀₋ₜ is 80-100% of marketed injectable compositions but Tmax is similar wherein similar Tmax means 100±10% of Tmax of currently marketed subcutaneous injectable compositions.

The present invention provides a composition comprising Ketamine salt equivalent to Ketamine base 50 mg/ml, a stabilizer and at least one pharmaceutically acceptable excipient. The composition exhibits lower Cmax, but similar Tmax and similar AUC as that of conventional Ketamine Injectable composition.

The composition comprises stabilizer selected from but not limited to pyrrolidone potassium (PVPK), Diethylene Glycol Monoethyl Ether or alkyl derivatives thereof, Polyethylene Glycol (PEG), Sodium carboxymethyl cellulose. In one embodiment the stabilizer is PVPK. In yet another embodiment, the stabilizer is PVPK-12 or PVPK-30.

In one embodiment, the composition comprises Ketamine and stabilizer in a ratio of 12:1 to 2:1. In one embodiment, the ratio of Ketamine and stabilizer is 10:1 to 3:1. In another embodiment, the ratio of Ketamine and stabilizer is 7:1 to 3:1.

In another embodiment, ratio of Ketamine to stabilizer is from 12.5:1 to 2.5:1, preferably from 10:1 to 3.33:1 and more preferably from 7.14:1 to 3.33:1.

The composition comprises at least one pharmaceutically acceptable excipient selected from but not limited to aqueous and or non-aqueous solvents, co-solvents, solubilizers, anti-oxidants, buffers/pH adjusting agents, chelating agents, preservatives, antimicrobials, and tonicity adjusting agents.

In one embodiment, the composition comprises chelating agent. The chelating agent is selected from but not limited to disodium EDTA, sodium EDTA, calcium disodium EDTA, versetamide, calteridol calcium, and diethylenetriaminepenta acetic acid. In one preferred embodiment, the chelating agent is disodium edetate.

The ratio of stabilizer to chelating agent in the composition is preferably 40:1 to 250:1. In one preferred embodiment the ratio is 50:1 to 160:1. In another preferred embodiment, the ratio is 50:1 to 150:1.

In yet another embodiment, the composition comprises isotonicity agent. In one preferred embodiment, the isotonicity agent is sodium chloride. The ratio of stabilizer to isotonicity agent in the composition is preferably 2.5:1 to 14.29:1. In one preferred embodiment the ratio is 2.94:1 to 9.38:1. In another preferred embodiment the ratio is 3.68:1 to 9.38:1.

In another preferred embodiment, the composition further comprises a preservative. The preservative is selected from but not limited to benzethonium chloride, benzyl alcohol, benzalkonium chloride, chlorobutanol, m-Cresol, methyl paraben, propyl paraben, and phenol. In one preferred embodiment, the preservative is benzethonium chloride. The amount of preservative in the composition is 0.1 mg/ml

In one preferred embodiment, the present invention provides a composition comprising Ketamine salt equivalent to Ketamine base 50 mg/ml, a stabilizer, a chelating agent, and an isotonicity agent. In one embodiment the composition comprises a stabilizer from 4-20 mg/ml, chelating agent from 0.05 to 0.2 mg/ml, and an isotonicity agent from 1.2-2 mg/ml. In another embodiment, the stabilizer is selected from povidone K12 and or K30, chelating agent is disodium edetate, and isotonicity agent is sodium chloride. In another embodiment the composition further comprises 0.10 mg/ml benzethonium chloride.

In one preferred embodiment the present invention provides an aqueous injectable composition comprising Ketamine salt equivalent to Ketamine base 50 mg/ml, povidone K12 15 mg/ml, disodium edetate 0.10 mg/ml, sodium chloride 1.6 mg/ml, and benzethonium chloride 0.10 mg/ml.

In another embodiment, the composition of present invention eliminates at slower rate than currently marketed Ketamine injectable compositions.

In yet another embodiment, the composition of present invention is administered by intramuscular and intravenous routs.

In still another embodiment, the present invention provides use of a composition comprising Ketamine salt equivalent to Ketamine base 50 mg/ml, a stabilizer and at least one pharmaceutically acceptable excipient for the treatment resistant depression and management of pain.

In yet another embodiment, the present invention provides a method for the treatment of treatment resistant depression and management of pain, wherein the method comprising administering a composition of any of the preceding claims.

In yet another embodiment, the composition provided in the present invention is a subcutaneous injectable composition. The composition is provided in a pack size of 50 mg Ketamine base/ml. Said pack size is equivalent to pack sizes such as 30 mg Ketamine base in 0.6 ml, 35 mg Ketamine base in 0.7 ml and 40 mg Ketamine base in 0.8 ml

The composition of present invention exhibits lesser or lower Cmax as compared to the currently marketed compositions indicating that the risk of concentration dependent-side effects is less with compositions of present invention.

In one embodiment, the composition exhibits the mean Cmax of 454.911 ng/mL which is well above the concentration needed for therapeutic efficacy in patients with depression (0.5 mg/kg; 40 min infusion; 185 ng/ml).

In another embodiment, the invention provides method of preparation of a composition as claimed in any preceding claims, wherein the method comprises obtaining a solution dissolving chelating agent, stabilizer, isotonicity agent, optionally a preservative, and Ketamine salt in specific amounts in water having dissolved oxygen content 0.5 mg/ml; and adjusting pH of the solution to 3.0 to 5.5, preferably 3-5.

In yet another embodiment, the Cmax of the composition is 80-100% of Cmax of the marketed injectable composition for subcutaneous use and AUC₀₋ₜ is 80-100% of marketed injectable composition, wherein Tmax is 100±10% of Tmax of currently marketed subcutaneous injectable composition.

### DETAILED DESCRIPTION OF THE INVENTION

The present invention relates to subcutaneous injectable pharmaceutical compositions comprising Ketamine or its salt such as hydrochloride and stabilizer with at least one pharmaceutically acceptable excipient. The blood levels exhibited by the composition of the present invention, upon administration render it free from at least one of the undesired effects. The pharmacokinetic profile of the composition of present invention was surprising. It was found that the composition of present invention has similar Tmax, but lower Cmax as compared to the conventional Ketamine 50 mg/ml injectable compositions, However, AUC of the present composition and currently marketed injectable compositions of Ketamine was same.

By way of definitions, in the present invention, (a) Cmax means the highest concentration of a drug in the blood, after a dose is given, (b) Tmax means the time it takes for a drug to reach the maximum concentration (Cmax) after administration of a drug and (c) AUC meaning Area under curve which means the area under the plot of Blood/plasma concentration of a drug versus time after dosage. It represents the total drug exposure across time. Two compositions are considered to be bioequivalent if the average bioavailability of the test composition is within (80% to 125%) that of the reference composition, with a certain assurance or if the estimated 90% confidence interval for the ratio of geometric means of the primary PK parameters (AUC and Cmax) is totally within the bioequivalence limits of 80% to 125%.

Lambda z/Terminal elimination rate constant—It describes the rate at which a drug is removed from the system/body. It is equivalent to the fraction of a substance that is removed per unit time measured at any particular instant. In Example 7, the value 0.4 for Test formulation compared to 0.5 for Reference formulation indicates test composition is eliminated at slower rate than reference formulation and hence test formulation remains for longer duration in body.

MRT INF_obs: (Observed Mean Residence time)—MRT obs represents the average time a molecule stays in the body. In Example 7, Value is 2 for Test composition, whereas 1.9 of Reference composition indicates test compound remains for longer time in body compared to reference formulation.

Mrt Last—MRT represents the average time a molecule stays in the body when observed for 24 hrs. In Example 7, Value is 2.1 for Test composition, whereas 2.0 of Reference composition indicating test composition remains for longer time in body compared to reference composition.

Vss obs is apparent volume of distribution at steady state. The volume of distribution (Vd) is a pharmacokinetic parameter representing an individual drug's propensity to either remain in the plasma or redistribute to other tissue compartments. A drug with a high Vd has a propensity to leave the plasma and enter the extravascular compartments of the body. Conversely, a drug with a low Vd has a propensity to remain in the plasma. Example 7 indicates that test formulation (Vss-0.0018) has more distribution in body compartment compared to reference formulation (Vss-0.0014).

AUC_% Extrap obs-AUC_{0-inf}: area under the concentration-time curve from time 0 to infinite time (extrapolated from available conc.)

AUC₀₋ₜ: Area under the quantifiable concentration-time curve from time 0 to the time of the last quantifiable concentration (here 24 hrs).

The present invention provides clear, stable subcutaneous injectable compositions comprising Ketamine or salts thereof equivalent to Ketamine base up to 50 mg/ml and a stabilizer along with at least one pharmaceutically acceptable excipient selected from but not limited to aqueous and or non-aqueous solvents, cosolvents, solubilizers, anti-oxidants, buffers/pH adjusting agents, chelating agents, complexing agents, preservatives, antimicrobials, tonicity adjusting agents.

It was surprisingly noticed that some carefully selected pharmaceutically acceptable ingredients enable the composition to exhibit reduced Cmax, similar or different Tmax and similar AUC and thereby get rid of at least one undesired effect of currently marketed injections without compromising efficacy when a stabilizer is present along with Ketamine. These substances or pharmaceutically acceptable ingredients which are essential for the compositions of the present invention and which cause favorable change in blood levels of Ketamine when compositions of the present invention are injected are termed as stabilizers. Incorporation of these substances enable compositions of the present invention to exhibit AUC similar to currently available compositions but do not manifest at least one of the undesirable effects or side effects as the Cmax of compositions of the present invention is lower while Tmax is similar. Such an amazing property of compositions of the present invention enables them to be used by patients and it reduces dependence on strict use by physician if allowed by law of the land. It also enables to make use of fullest medicinal potential of the Ketamine molecule. Polyethylene glycol of various grades such as PEG 300, PEG 400 or pharmaceutically acceptable different oils may also be used as Stabilizers. Present invention is illustrated using PVPK as stabilizer. PVPK is or povidone K or Polyvinylpyrrolidone (PVP) potassium, commonly called polyvidone, is a water-soluble polymer made from the monomer N-vinylpyrrolidone. PVPK is available in a range of molecular weights and related viscosities. Various grades of PVPK are useful to perform the invention and the invention is illustrated using PVPK-12 and PVPK30 grade. Diethylene Glycol Monoethyl Ether or alkyl derivatives thereof, Polyethylene Glycol (PEG), Sodium CMC can also be used as Stabilizers. The most critical ratio governing compositions of the present invention is that of Ketamine:Stabilizer. Ratio of Ketamine:Stabilizer from 12.5:1 to 2.5:1, more preferable being 10:1 to 3.33:1 and the most preferable ratio is 7.14:1 to 3.33:1 is necessary to prepare compositions of present invention where although Cmax is lower, AUC is similar.

One such exemplary subcutaneous injectable composition comprising Ketamine and a stabilizer having 3 Liter batch size is stated below. Quantity of ingredients is expressed as mg/ml for brevity and the process to prepare it is described:

| Sr. No. | Item Name | Quantity |
|---|---|---|
| A. | Raw Material | (mg/mL) |
| 1 | Ketamine hydrochloride USP | 50.00 |
| | equivalent to Ketamine base | |
| 2 | Povidone K12 BP | 4.00 |
| 3 | Sodium chloride BP | 1.60 |
| 4 | Disodium Edetate BP | 0.10 |
| 5 | Benzethonium chloride USP | 0.10 |
| 6 | Water For Injection USP | q.s. to 1 mL |

Quantity of Ketamine is to be calculated based on Assay and LOD.

Process to prepare the compositions of the invention is described below:
1. Check the weight of all the ingredients as per dispensing sheet and calculation sheet. Check the tag label for the status of the materials.
2. Identify the SS manufacturing tank with in-process label.
3. Collect about 4 L of water for injection in jacketed mfg. tank from circulating loop. Start continuous stirring and nitrogen bubbling/sparging and overlaying. Check the dissolved oxygen content of water for injection by using calibrated Dissolved Oxygen (DO) meter (DO should not more than 0.5 mg/L). After checking DO, transfer 2 L water for injection from the jacketed manufacturing tank to a separate SS vessel and start nitrogen bubbling/sparging. Keep aside for volume adjustment. Cool the water for injection in jacketed manufacturing tank and SS vessel to 50 °C to 60 °C.
4. Add and dissolve dispensed quantity of Disodium edetate (a chelating agent) followed by Povidone K12 (a stabilizer) in the jacketed mfg. tank. Rinse the container of Disodium edetate two times with water for injection from SS vessel and add it in jacketed mfg. tank. This is chelating agent solution.
5. Add and dissolve dispensed quantity of Sodium Chloride (isotonicity agent) in above jacketed mfg. tank. Rinse the container with water for injection and add it in jacketed manufacturing tank under stirring and nitrogen bubbling/sparging and overlaying by stirring for 10-15 minutes to prepare isotonic solution.
6. Add and dissolve dispensed quantity of Benzethonium chloride (preservative) in jacketed mfg. tank. Rinse the container with water for injection and add it in jacketed manufacturing tank under stirring and nitrogen bubbling/sparging and overlaying by stirring till a clear solution is obtained. This is preservative solution. This step is optional and not applicable in case of compositions where preservative is not used
7. Cooled the above solution isotonic solution obtained in step 5 or preservative solution obtained in step 6 up to room temperature by applying chilled water in the jacket and nitrogen purging. Checked the temperature of solution. This is cooled solution.
8. Add and dissolve dispensed quantity of Ketamine hydrochloride in jacketed manufacturing tank in cooled solution obtained in step 7 with continuous stirring and nitrogen purging. Rinse container with water for injection and add it to jacketed mfg. tank under stirring and nitrogen bubbling/sparging and overlaying by stirring to obtain a clear solution. This is Ketamine solution and checked clarity of solution.
9. Make up the volume Ketamine solution obtained in step 8 to approximately 2.5 L with water for injection. Stir the solution for 10 minutes with nitrogen bubbling/Sparging and overlaying. Check the pH of solution of jacketed manufacturing tank. Limit of pH to 3.0 to 5.0. This is pH tested solution.
10. Make up the final volume of pH tested solution obtained in step 9 up to 3 Liters using remaining quantity of water for injection from SS container to prepare unfiltered solution. Finally stir the unfiltered solution for 30 minutes with nitrogen bubbling/Sparging and overlaying. This is bulk solution.
11. Collect the sample of bulk solution into glass bottle and send it to Quality Control Department with in-process test request for bulk analysis.
12. Close all opening of jacketed manufacturing tank and Blanket the manufacturing tank with nitrogen bubbling and complete the filtration.

Filtration is carried out to sterilize the composition by filtering through 0.22 mu filter. Solvent in the present invention is water for injection. One may use suitable quantities of other aqueous or non-aqueous solvents such as Diethylene Glycol Monoethyl Ether (Transcutol) or alkyl derivatives thereof or various grades of Glycofurol. Commonly used solvents such as water, ethanol can be employed to perform the invention. Various types of alcohols can be employed as solvents and or solubilizers. Alcohols selected from monohydric alcohol or polyhydric alcohols may be employed. An alcohol other than monohydric alcohol is to be interpreted as polyhydric alcohol. Examples of monohydric alcohol are any pharmaceutically acceptable single chain or branched chain alcohol having only one OH group. Suitable examples are ethyl alcohol or benzyl alcohol. Glycerin is example of polyhydric alcohol. There are many other polyhydric alcohols. One may use polyethylene glycol or some ether derivatives as good solubilizers.

Terms Solvents, Cosolvents or solubilizer are used interchangeably. These can be solids or liquids. Solids when present in the form of solution in appropriate solvents also act as solubilizers. Ketamine salt used in the present invention and other ingredients used in the present invention are water soluble.

Solubilizers may be selected from but not limited to Transcutol 2 to 50%, PEG 300 up to 50%, PEG 400 up to 20.30%, Polysorbates or Tweens such as Tween 80. It may also comprise suitable vegetable oils as solvents. Alcohols such as Ethanol are good solubilizer. The concentration of Diethylene Glycol Monoethyl Ether or alkyl derivatives thereof in range 0.2%-5% (v/v) act as solubilizers as well as stabilizers either alone or in combination with other stabilizers.

The invention provides therapeutically effective amount of Ketamine or salts such as hydrochloride salt in an aqueous composition suitable to be administered by subcutaneous route.

Composition of the present invention is ready-to-use composition of Ketamine or salts thereof in management of pain and treatment of treatment resistant depression.

In particular, the invention provides a simple and economical composition comprising Ketamine or its salts such as hydrochloride salt thereof equivalent to Ketamine base 50 mg/ml suitable for subcutaneous administration that is bioequivalent with conventionally available injectable compositions comprising Ketamine base 50 mg/ml.

Compositions of the present invention exhibit viscosity of 1.0 cps to 3.0 cps.

Glycofurol of various grades such as Glycofurol 75 up to 5%, may be used as solvent or co-solvent or solubilizer. Tweens such as Tween 80 acts as surfactant or solubilizer.

The present invention optionally uses antioxidants selected from but not limited to thioglycerol, acetyl cysteine, butylated hydroxy anisole (BHA) and butylated hydroxy toluene (BHT), ascorbates, ascorbyl palmitate, methyl paraben, propyl paraben, thiomerosal and mixed Tocopheryl ingredient. Further, the invention may also comprise additional excipients like preservatives such as benzalkonium chloride, benzyl alcohol, m-cresol, parabens, phenols, thiomerosal. In another embodiment the composition of present invention may also comprise the stabilizers such as polysorbate 80, l-cysteine, diethanolamine, 1-methionine, sodium gluconate, sodium thioglycolate, triethanolamine and oleic acid. These are to be incorporated as per guidelines allowing their incorporation with respect to quantity and applicable specifications known to persons skilled in the art.

The isotony of the composition is obtained by adding a precisely calculated quantity of isotonicity agent or tonicity adjusting agent selected from sodium chloride, dextrose anhydrous, Glycerin, glucose, mannitol, sorbitol, potassium chloride or calcium chloride. The most preferred isotonic agent is sodium chloride. The ratio of stabilizer to isotonicity agent is 2.5:1 to 14.29:1, preferably being 2.94:1 9.38:1 and the more preferably being 3.68:1 to 9.38:1 The compositions of present invention having osmolality from about less than 100 to about 800 mOsm/kg. The solution isotony or isotonicity is between 100 and 400 mOsm/kg is desirable and obtained. Techniques and processes to adjust the isotonicity or osmolality are known in the art and accordingly isotonicity should be adjusted.

The composition of present invention comprises chelating agents. The chelating agents that may be used in formulation to chelate traces of metallic impurity may be selected from but not limited to disodium EDTA, sodium EDTA, Calcium disodium EDTA 0.2%, Versetamide 2.54%, Calteridol Calcium 0.023%, also Diethylenetriaminepenta Acetic Acid and ingredients alike. In one embodiment the ratio of stabilizer to chelating agent in the composition is 40:1 to 250:1, preferably 50:1 to 160:1 and the more preferably 50:1 to 150:1. The ratio of stabilizer to chelating agent is critical for the compositions.

In some of the embodiments, the composition comprises antimicrobial preservatives. In other embodiments, the composition does not have any preservatives. The preservatives when present are selected from but not limited to benzethonium chloride 0.01%, benzyl alcohol up to 2%, benzalkonium chloride 0.02%, chlorobutanol 2.5 to 5%, m-cresol 0.1% to 0.3%, parabens like methyl paraben, propyl paraben up to 1%, phenol up to 0.45%. Some more excipients like 2-PhenoxyEthanol, Phenyl Mercuric Nitrate, Thiomersal and excipients alike or mixtures thereof. Benzyl alcohol acts as solvent also. In preferred embodiments the preservative used is benzethonium chloride.

Suitable surfactants such as Polysorbates/Tweens, sorbitan mono laurate, lecithin, povidone or other pharmaceutically acceptable surfactants may be used either individually or in combination to prepare compositions of the invention.

A pH close to the physiological one is recommended to minimize pain, irritation, and tissue damage. Buffers optimize solubility and stability by adjusting the pH.

Examples of pH adjustment agents or buffers that may be employed include but not limited to sodium, potassium or ammonium salt of a weak acid, a tris-(hydroxymethyl)-aminomethane, or Sodium citrate, Sodium phosphate, Sodium Hydroxide, Tris base-65, Tris acetate, Tris HCl-65 or commonly known acetates, citrates, phosphates or a further physiologically active material acting as a buffer such as sodium hydroxide, hydrochloric acid, boric acid, citric acid, acetic acid, phosphoric acid, succinic acid, potassium hydroxide, ammonium hydroxide, magnesium oxide, calcium carbonate, magnesium carbonate, malic acid, potassium citrate, sodium phosphate, lactic acid, gluconic acid, tartaric acid, fumaric acid, diethanolamine, monoethanolamine, sodium carbonate, sodium bicarbonate, triethanolamine, or any combination thereof. The stable aqueous injectable solution may comprise one or more pH adjusting agents in sufficient quantity to provide pH of the solution between about 3 and about 5.5 preferred being between 3 to 5 during storage.

The compositions are filled, sealed, packed and provided in suitable containers made up of glass or plastic preferably of single compartment which may be composed of low density polyethylene (LDPE), high density polyethylene (HDPE), polypropylene (PP) or mixtures of polyethylene and polypropylene. Preferably used glass material is USP type I glass (borosilicate glass) and type II glass. In another embodiment, the said composition may be provided in volume of less than 1 ml of single compartment or ready-to-use injection which can be diluted according to physiological use. Containers used to contain compositions of present invention may be stoppered by rubber stoppers, closures or disc seals, screw-caps or cap-stopper combination seals closures.

The compositions are subjected to sterilization process to achieve the sterile compositions. The formulation is prepared and filled in article and is terminally sterilized by electron beam irradiation such as T-irradiation, natural light, microwave heat viz. moist heat sterilization, dry heat sterilization. Autoclave sterilization may be preferred to achieve moist heat sterilization after final packaging. Typical cycle of autoclave to attain the sterilization of the final product is 121 °C for minutes.

The compositions can be autoclaved at temperature ranging from 110 °C to 130 °C, preferably 115 °C to 125 °C for period of time ranging from 5 minutes to 30 minutes, preferably 10 minutes to 20 minutes. More preferably the autoclaving is done at 120 °C to 122 °C for 15 minutes.

The compositions after sterilization were stored at different stability conditions as per the stability guidelines. The compositions provide an accurate dose active.

In another embodiment, composition is having pH ranging from 3.00 to 5.00 and viscosity is in between 1.0 cps to 3.0 cps.

The injectable compositions of the present invention exhibit AUC similar to that of currently marketed injectable compositions of Ketamine although Cmax is lower and Tmax is more or less similar. The undesired effects associated with currently marketed injectable compositions of Ketamine are not associated with compositions of present invention although AUC is similar. The compositions do not cause sharp spike of Ketamine as is caused by conventional compositions and hence are safe and effective. The compositions of the present invention are suitable for administration by subcutaneous route.

The most critical ratio governing compositions of the present invention is that of Ketamine: Stabilizer. Ratio of Ketamine: stabilizer from 12.5:1 to 2.5:1, more preferable being 10:1 to 3.33:1 and the most preferable ratio is 7.14:1 to 3.33:1.

It is better to keep ratio of Stabilizer to Isotonicity agent 2.5:1 to 14.29:1, more preferable being 2.94:1 9.38:1 and the most preferable being 3.68:1 to 9.38:1.

It is better to keep ratio of Stabilizer:Chelating agent 40:1 to 250:1, more preferable being 50:1 to 160:1 and the most preferable being 50:1 to 150:1.

Thus are provided subcutaneous injectable compositions comprising Ketamine equivalent to Ketamine base 50 mg/ml with stabilizer which exhibit lesser Cmax as compared to the currently marketed compositions indicating that the risk of concentration dependent-side effects is less with compositions of present invention.

Thus there are provided subcutaneous injectable compositions comprising Ketamine equivalent to Ketamine base 50 mg/ml with stabilizer which exhibit Mean Cmax of 454.911 ng/mL which is well above the concentration needed for therapeutic efficacy in patients with depression.

Thus there are provided subcutaneous injectable compositions comprising Ketamine equivalent to Ketamine base 50 mg/ml with stabilizer which exhibit same Tmax as that of the currently marketed formulation meaning the efficacy of onset of action is not compromised or delayed but is similar to that of marketed compositions.

Clear solutions and immediate release injectable compositions are considered bioequivalent unless they contain some ingredient that can impact release and hence bioavailability of active. There are all chances that release and hence bioavailability of Ketamine would be altered and it would be rendered not bioequivalent due to addition of ingredient not present in conventional injectable compositions. Compositions of the present invention comprise of stabilizer, an ingredient i.e. PVPK or PEG and other listed here before that have capability of rendering it not bioequivalent. The invention also resides in specific quantities of stabilizer that ensures the compositions of the present invention are bioequivalent yet free from side effects associated with conventional injectable compositions because surprisingly it lowers the Cmax, does not impact Tmax and surprisingly maintains similar AUC.

It may be noted that Cmax and AUC of compositions of the present invention ie Test compositions falls within the criteria of 80%-125% of that of Reference composition. Thus compositions of the present invention are bioequivalent to currently marketed compositions.

The above and other objects, features and advantages of the present invention will now be described in detail with reference to certain exemplary embodiments thereof illustrated in the accompanying examples but are not limitative of the present invention. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims. Thus composition wherein an additional active ingredient is present along with claimed % of Ketamine and or ratios of Ketamine:Stabilizer would fall within the purview of the present invention.

While the present invention has been described in terms of a few specific aspects, modifications and equivalents thereof, in light of teaching and disclosure of the present invention, which are apparent to the skilled artisan, are to be construed as included within the scope of the invention.

### EXAMPLES

### Example 1: Ketamine Subcutaneous Composition

Batch size of 3 Liters was prepared for Trial A and for Trial G. Quantity expressed as mg/ml.

| Item Name | A(mg/ml) | G(mg/ml) |
|---|---|---|
| Ketamine hydrochloride | 50.00 | 50.00 |
| USP = Ketamine base | | |
| Povidone K12 BP | 4.00 | 15.00 |
| Sodium chloride BP | 1.60 | 1.60 |
| Disodium Edetate BP | 0.10 | 0.10 |
| Benzethonium chloride USP | 0.10 | 0 |
| Water For Injection USP | q.s. to 1 mL | q.s. to 1 Ml |

### Example 2: Procedure to Prepare Composition in Example 1

1. Checked the weight of all ingredients as per dispensing sheet and calculation sheet. Checked tag labels for the status of the materials.
2. Identified the SS manufacturing tank with in-process label.
3. Collected about 4 L of water for injection in jacketed mfg. tank from circulating loop. Started continuous stirring and nitrogen bubbling/sparging and over laying. Checked dissolved oxygen content of water for injection by using calibrated DO (dissolved oxygen) meter (DO should not more than 0.5 mg/L). After checking DO, transferred 2 L water for injection from the jacketed manufacturing tank to a separate stainless steel (SS) vessel and started nitrogen bubbling/sparging and kept aside for volume adjustment. Cooled the water for injection in the jacketed manufacturing tank as well as in SS vessel to 50 °C to 60 °C.
4. Added and dissolved dispensed quantity of Disodium edetate followed by Povidone K12 in the jacketed manufacturing. tank. Rinsed the container of Disodium edetate two times with water for injection from SS vessel and added it in jacketed manufacturing tank. This is chelating agent solution.
5. Added and dissolved dispensed quantity of Sodium Chloride in above jacketed mfg. tank. Rinsed the container with water for injection and added it in jacketed mfg. tank under stirring and nitrogen bubbling/sparging and overlaying by stirring for 10-15 minutes to prepare isotonic solution.
6. Added and dissolved dispensed quantity of Benzethonium chloride (In case of Trial A) in jacketed mfg. tank. Rinsed the container with water for injection and added it in jacketed mfg. tank under stirring and nitrogen bubbling/sparging and overlaying by stirring till a clear solution was obtained. This is preservative solution. This step is optional and not applicable in case of compositions where preservative is not used such as Trial G.
7. Cooled the isotonic solution obtained in step 5 or preservative solution obtained in step 6 up to room temperature by applying chilled water in the jacket and nitrogen purging to bring down the temperature to 40 °C-45 °C. This is cooled solution.
8. Added and dissolved dispensed quantity of Ketamine hydrochloride in jacketed manufacturing tank in cooled solution obtained in step 7 with continuous stirring and nitrogen purging. Rinsed container with water for injection and added it to jacketed mfg. tank under stirring and nitrogen bubbling/sparging and overlaying by stirring till a clear solution is obtained. This is Ketamine solution and checked clarity of solution.
9. Made up the volume of Ketamine solution obtained in step 8 to approximately 2.5 L with water for injection and Stirred for 10 minutes with nitrogen bubbling/Sparging and overlaying and checked pH of solution. Limit of pH to 3.0 to 5.0. This is pH tested solution.
10. Made up the final volume of pH tested solution obtained in step 9 up to 3 L by using remaining quantity of water for injection from SS container to prepare unfiltered solution. Stirred the unfiltered solution for 30 minutes with nitrogen bubbling/Sparging and overlaying. This is bulk solution.
11. Collected sample of bulk solution obtained in step 10 into glass bottle and sent it for bulk analysis.
12. Closed all opening of jacketed manufacturing tank and Blanketted the manufacturing tank with nitrogen bubbling till filtration is completed.
13. Filtration is carried out to sterilize the composition by filtering through 0.22 mu filter to prepare filtered or sterilized solution.
14. Filled and sealed aseptically the filtered or sterilized solution into ampoules and vials and subjected to stability studies.

### Example 3: Compositions with Varying Stabilizer Quantities

Quantity of each ingredient is expressed as (mg/mL)

| Item Name | B | C | D | E | F |
|---|---|---|---|---|---|
| Ketamine hydrochloride | 50.00 | 50.00 | 50.00 | 50.00 | 50.00 |
| USP = Ketamine base | | | | | |
| Povidone K12 BP | 8 | 20 | 5 | 7 | 7 |
| Sodium chloride BP | 1.2 | 1.4 | 1.7 | 1.9 | 2 |
| Disodium Edetate BP | 0.05 | 0.08 | 0.1 | 0.15 | 0.2 |
| Benzethonium chloride USP | 0.1 | 0.1 | 0.1 | 0.1 | 0.1 |
| Water For Injection USP | 1 | 1 | 1 | 1 | 1 |

### Example 4

Analysis and Assay of Ketamine compositions was performed as per method of analysis described in IP-2022, Volume-II, Page no. 2667-2668.

### Example 5

Example 1 composition G-6 Month Stability at 25 °C±2 °C/60%±5% RH

| Test | Specification | Initial | 6 Month |
|---|---|---|---|
| Description | A Clear, colourless solution | Complies | Complies |
| PH (at 25 °C) | 3.0 to 5.5 | 3.61 | 4.44 |
| Assay (By | (NLT 47.5 and NMT 52.5) mg | 100.34% | 98.44% |
| UV) | of the stated amount. (NLT | | |
| | 95.0% and NMT 105.0% of LC) | | |
| Related | Any single highest Impurity not | Below | Below |
| Substance | more than (NMT) 0.5% | detection | detection |
| | Any other highest impurity | limit. | limit. |
| | NMT 0.25% | | |

### Example 6

Example 1 Composition G: 6 Month Stability at 40 °C±2 °C/75%±5% RH

| Test | Specification | Initial | 6 Month |
|---|---|---|---|
| Description | A Clear, colourless solution | Clear, | Clear, |
| | | colourless | colourless |
| | | solution | solution |
| PH (at 25 °C) | 3.0 to 5.5 | 3.61 | 4.60 |
| Assay (By | (NLT 47.5 and NMT 52.5) | 100.34% | 98.13% |
| UV) | mg of the stated amount | | |
| | (NLT 95.0% and NMT 105.0% | | |
| | of LC) | | |
| Related | Any single highest | Below | Below |
| Substance | Impurity NMT 0.5% | detection | detection |
| | Any other highest | limit. | limit. |
| | impurity NMT 0.25% | | |

### Example 7: Pharmacokinetic Parameters (Mean±SD) of Ketamine Test Product and Reference Marketed Formulation

| | Test | Reference |
|---|---|---|
| Cₘₐₓ | 454.9 | 554.5 |
| AUC_{0 to t} | 693.0 | 847.0829846 |
| AUC_{0 to inf}_obs | 699.6813577 | 851.9259781 |
| AUC_% Extrap obs | 6.7 | 4.843 |
| Lambda_z | 0.4 | 0.5 |
| MRTINF_obs | 2 | 1.9 |
| MRT last | 2.1 | 2 |
| Tlast | 24 | 24 |
| Tₘₐₓ | 0.5 | 0.5 |
| Vss_obs | 0.0018 | 0.0014 |

It may be noted that Cmax of Test composition is 82.04% of Cmax of reference product.

Tmax of Test and Reference composition is identical.AUC₀₋ₜ of Test composition is 82.129% of Reference composition. The value 0.4 of Lambda_z for Test composition compared to 0.5 for Reference composition indicate test composition is eliminating at slower rate than reference composition and hence test composition remains for longer duration in body. Value 2 of MRTINF_obs for Test composition, and 1.9 for Reference composition indicates test composition remains for longer time in body compared to reference composition. Value of MRT last is 2.1 for Test composition, whereas 2.0 for Reference composition indicating test compound remains for longer time in body compared to reference composition. Values of Vss_obs indicate that Test composition (Vss—0.0018) has more distribution in body compartment compared to reference composition (Vss—0.0014). T last (Time of Last observed concentration) indicates concentrations of analyte at 24 hrs (last timepoint of blood collection.)

### Example 8

Compositions with PEG-400, PVPK-30, PVPK-12: A Composition with PEG-400, PVPK-30, PVPK-12 was prepared by a process of example 2. In one composition PEG-400 was incorporated in an amount of 0.2 ml/ml of composition. In another composition PVP K-30 was incorporated in an amount of 5.9 mg/ml of composition. In yet another composition PVPK-12 was incorporated in an amount of 10 mg/mL. Other ingredients remaining same. Formulations were found stable.

### Example 9: Study of Blood Concentrations of Ketamine in Rats

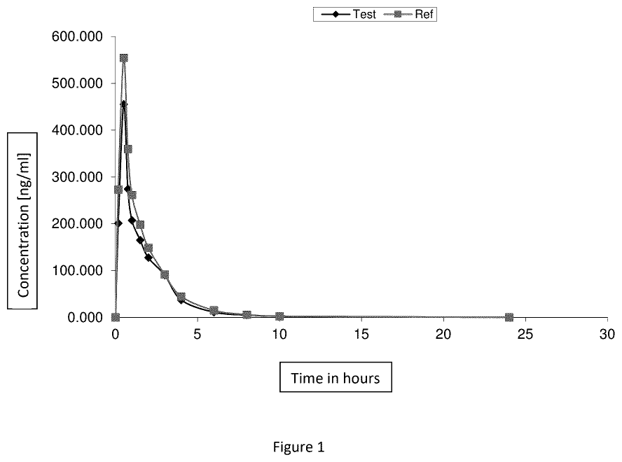

FIG. 1 provides a graph of blood concentrations of Ketamine in nanograms (ng)/ml on Y axis from Test and Reference compositions against time in hours on X axis.

24 rats (12 males and 12 females) were randomly allocated based on body weights to 2 different groups G1 and G2, each having 6 animals/sex. Following randomization, selected animals were identified by animal number written on base of tail.

For G1 and G2 groups, dose formulation was prepared to achieve concentration of 3.1 mg/ml. e.g. 1 ml of Composition of the invention was diluted to 16 ml of normal saline to give concentration of 3.1 mg/ml. Composition of the invention required for 200 g Rat (3.1 mg/kg dose) is 0.62 mg. (3.1*200/1000=0.62 mg) To administer 0.62 mg of composition of invention of 3.1 mg/ml concentration, 0.2 ml of dose volume required to be administered to a rat. The dose most commonly used in Humans is 0.5 mg/kg. The conversion factor for Rat is 6.17. So Rat equivalent dose is 3.1 mg/kg.

Animals in G1 were administered Composition of the invention and animals in G2 were administered Ketamine 50 Injection manufactured by Themis Medicare Ltd, a marketed formulation to receive 3.1 mg/kg body weight by subcutaneous route.

Blood collection was done from retro-orbital sinuses. Ketamine is dissociative anaesthetic agent and hence initial blood collection was done without using any anaesthetic agent and isofluorane was used at subsequent blood collection time points. Approximately 0.2 ml to 0.3 ml blood was collected at each time point in vials containing 1% EDTA as an anticoagulant followed by separation of plasma for analysis. Separated plasma was transferred to prelabelled polypropylene tubes and stored at −80 °C until transferred to analytical laboratory for bioanalysis by standard procedures.

The peak plasma concentrations (Cmax) for Test and Reference were found to be 454.911 ng/mL and 554.509 at 0.50 hour and 0.50 (Tmax) hours respectively [FIG. 1]. The time to attain the Cmax for Test and Reference were found to be similar indicating that both the test formulations exhibit similar release and absorption profile. The observed plasma concentration at 24 hour (C-24 hr) in Test and Reference were 0.000, 0.000 ng/mL, respectively.

The AUC was determined to understand the total exposure of the Ketamine concentrations to be remained in the blood, which will determine the dosing of the compounds. In the present study, AUC_{0 to t} for Test and reference formulation was found 692.979 ng/mL and 847.083, respectively.

### Example 10

Behavioural testing comparing compositions of the invention with presently marketed Ketamine 50 injection 5 ml vial, Manufactured by Themis Medicare Limited (Generic name: Ketamine Hydrochloride Injection IP 50 mg/ml)

A study was conducted to evaluate the effects on the behavior in Sprague Dawley rats when administered by subcutaneous route of composition of the invention in comparison with Ketamine 50 Injection Marketed Formulation.

Animals were divided into three groups each group containing 6 animals. G1 group was control group to whom normal saline was administered. To animals in G2 group, Composition of invention was administered and to group G3 marketed formulation was administered, subcutaneously in 3 doses ie 1.5 mg/kg, 3 mg/kg and 6 mg/kg on three different days in ascending manner.

Dose volume did not exceed 1 mL/kg. Actual volume administered was calculated based on the recent body weight of each animal.

Animals were evaluated for Open field test, Learning and memory test (e.g. Y maze Test) and Functional observational battery test.

### Open Field Test:

1) At 1.5 mg/kg Number of rears were reduced in G2 group but the reduction was more in G3. It indicates Composition of the present invention have lesser inhibitory effect of locomotion and movements and that Composition of the present invention is better than the currently marketed formulation in terms of less deterioration of locomotor activity at a dose of 1.5 mg/kg in rats.
Urination: Number of urinations slightly increased in G3 group than in G2 group animals. It means G2 animals have possibly better muscle control than animals in G3. So at 1.5 mg/kg, Composition of the present inventions have lesser side effects or is devoid of some effects present in marketed compositions.
2) At 3 mg/kg Incidence of ataxic gait (a failure of muscle coordination) was more in G3 group males and females than of G2 group. It means at 3 mg/kg dose, the disturbance of muscle coordination was less in the Composition of the present invention group ie G2 than the marketed formulation ie G3.
3) At 6 mg/kg number of rears were more reduced in G3 group animals than those of G2 group animals. It means at 6 mg/kg dose, the disturbance of muscle coordination was less in the Composition of the present invention group ie G2 than the marketed formulation ie G3.

At 3 mg/kg and 6 mg/kg G2 animals have better muscle control than animals in G3 meaning compositions of the present inventions enable better muscle coordination and control than marketed compositions.

### Learning and Memory Test:

At 1.5 mg/kg, spatial reference memory (SRM) of G2 was better than G3. In G3 group it decreased markedly. At 3 mg/kg, G2 group males showed increase in same arm return (% SAR).

With respect to spatial reference memory (SRM test), composition of the present invention causes less deterioration of spatial memory as compared to the currently marketed formulation at low Dose (1.5 mg/kg) and Mid dose (3 mg/kg).

### Functional Observational Battery:

At 1.5 mg/kg dose, there was significant difference in paws distance in animals treated with Ketamine marketed formulation than with composition of the present invention, indicative of inability to righting appropriately on hind paws.

At 3.0 mg/kg, the incidence of righting reflexes was higher in G2 group than G3.

At 6.0 mg/kg, G2 and G3 were comparable with those of controls.

This indicates that muscle coordination and balance are less affected by Composition of the present invention as compared to Ketamine marketed formulation at lower dose. It also means that Composition of the present invention at 1.5 mg/kg dose has less potential for neurotoxicity as compared to the Ketamine marketed formulation.

### SUMMARY & CONCLUSION

This indicates Composition of present invention is better than the currently marketed ketamine injectable formulation in terms of less deterioration of
1. Locomotor activity at lower dose.
2. Spatial Reference Memory (SRM) at lower & middle doses
3. Muscle coordination and balance at lower dose.

### DEFINITIONS

Spatial reference memory (SRM) represent long-term memory responsible for spatial recognition (related to space). Spontaneous alteration performance (SAP) which is indicative of retrograde working memory and cognitive function. Same Arm Return (SAR) ratio estimates some aspects of attentional dysfunction within active working memory performance in the Y-Maze.

SAP indicates that the animal is having a good working memory will remember the arms of the maze that it has already visited and will show a tendency to enter a less recently visited arm.

SRM indicates intact spatial reference memory, animal enters and spend more time in the novel arm compared to the other arms of the maze. Animals with impaired spatial reference memory will is not be able to identify the novel arm.

1. A composition comprising Ketamine salt equivalent to Ketamine base 50 mg/ml, a stabilizer and at least one pharmaceutically acceptable excipient; wherein the composition exhibits lower Cmax, but similar Tmax and similar AUC as that of conventional Ketamine Injectable composition.

2. The composition as claimed in claim 1, wherein the composition comprises Ketamine and stabilizer in a ratio of 12:1 to 2:1.

3. The composition as claimed in claim 1, wherein the composition comprises Ketamine and stabilizer in a ratio of 10:1 to 3:1.

4. The composition as claimed in claim 1, wherein the composition comprises Ketamine and stabilizer in a ratio of 7:1 to 3:1.

5. The composition as claimed in claim 1, wherein the stabilizer is selected from pyrrolidone potassium (PVPK), Diethylene Glycol Monoethyl Ether or alkyl derivatives thereof, Polyethylene Glycol (PEG), Sodium carboxymethyl cellulose.

6. The composition as claimed in claim 1, wherein the stabilizer is polyvinyl pyrrolidone potassium (PVPK).

7. The composition as claimed in claim 1, wherein pharmaceutically acceptable excipient is selected from aqueous and or non-aqueous solvents, co-solvents, solubilizers, anti-oxidants, buffers/pH adjusting agents, chelating agents, preservatives, antimicrobials, and tonicity adjusting agents.

8. The composition as claimed in claim 1, wherein the excipient is a chelating agent selected from selected from disodium EDTA, sodium EDTA, calcium disodium EDTA, versetamide, calteridol calcium, and diethylenetriaminepenta acetic acid.

9. The composition as claimed in claim 1, wherein the excipient is a preservative selected from benzethonium chloride, benzyl alcohol, benzalkonium chloride, chlorobutanol, m-Cresol, methyl paraben, propyl paraben, and phenol.

10. The composition as claimed in claim 1, wherein the excipient is an isotonicity agent selected from sodium chloride, dextrose anhydrous, glycerin, glucose, mannitol, sorbitol, potassium chloride and calcium chloride sodium chloride.

11. The composition as claimed in claim 8, wherein the ratio of stabilizer to chelating agent is 40:1 to 250:1.

12. The composition as claimed in claim 10, wherein the ratio of stabilizer to isotonicity agent is 2.5:1 to 14.29:1.

13. The composition as claimed in claim 9, wherein the preservative is present in an amount of 0.1 mg/ml.

14. The composition as claimed in claim 1, wherein the composition is an aqueous subcutaneous injectable composition.

15. The composition as claimed in claim 1, wherein the composition is administered by intramuscular and intravenous routes.

16. The composition as claimed in claim 1, wherein the composition eliminates at slower rate than currently marketed Ketamine injectable compositions.

17. A composition comprising Ketamine salt equivalent to Ketamine base 50 mg/ml, a stabilizer in an amount from 4-20 mg/ml, chelating agent in an amount from 0.05 to 0.2 mg/ml, and an isotonicity agent in an amount from 1.2-2 mg/ml.

18. The composition as claimed in claim 17, wherein the stabilizer is selected from povidone K12 and K30, chelating agent is disodium edetate, and isotonicity agent is sodium chloride.

19. The composition as claimed in claim 17, wherein the composition further comprises benzethonium chloride in an amount of 0.1 mg/ml.

20. An aqueous injectable composition comprising Ketamine salt equivalent to Ketamine base 50 mg/ml, povidone K12 in an amount of 15 mg/ml, disodium edetate in an amount of 0.10 mg/ml, sodium chloride in an amount of 1.6 mg/ml, and benzethonium chloride in an amount of 0.10 mg/ml.

21. The composition as claimed in claim 20, wherein the mean Cmax exhibited by the composition is 454.91 ng/ml.

22. The composition as claimed in claim 1, wherein Cmax of the compositions is 80-100% of Cmax to the marketed injectable compositions for subcutaneous use and AUC0-t is 80-100% of marketed injectable compositions, wherein Tmax is 100±10% of Tmax of currently marketed subcutaneous injectable compositions.

23. A method of preparation of a composition as claimed in claim 1, wherein the method comprises obtaining a solution dissolving chelating agent, stabilizer, isotonicity agent, optionally a preservative, and Ketamine or salt thereof in a specific amount in water having dissolved oxygen content 0.5 mg/ml; and adjusting pH of the solution to 3.0 to 5.5.

24. (canceled)

25. A method for the treatment of treatment resistant depression and management of pain, wherein the method comprising administering the composition of claim 1.

26. The composition as claimed in claim 1, wherein the stabilizer is PVPK-12 or PVPK-30.

27. The composition as claimed in claim 1, wherein the excipient is the chelating agent disodium EDTA.

28. The composition as claimed in claim 1, wherein the excipient is the preservative benzethonium chloride.

29. The composition as claimed in claim 1, wherein the excipient is the isotonicity agent sodium chloride.

30. The composition as claimed in claim 8, wherein the ratio of stabilizer to chelating agent is 50:1 to 150:1.

31. The composition as claimed in claim 10, wherein the ratio of stabilizer to isotonicity agent is 3.68:1 to 9.38:1.

32. A method of preparation of a composition as claimed in claim 1, wherein the method comprises obtaining a solution dissolving chelating agent, stabilizer, isotonicity agent, optionally a preservative, and Ketamine or salt thereof in a specific amount in water having dissolved oxygen content 0.5 mg/ml; and adjusting pH of the solution to 3 to 5.
